SEQUENCE LISTING (3 sequences)

SEQ ID NO: 1 - protein, 6 aa, synthetic construct
  REGION 1..6: note = (S)-2-amino-N-(14-amino-2,5,8,11,14-pentaoxo-3,6,9, 12-tetraazatetradecyl)-3-(perfluorophenyl) propenamide
  MOD_RES 1: note = L perfluorophenylalanine
FGGGGG

SEQ ID NO: 2 - protein, 6 aa, synthetic construct
  REGION 1..6: note = 2-amino-N-(14-amino-2,5,8,11,14-pentaoxo-3,6,9,12-t etraazatetradecyl)acetamide
GGGGGG

SEQ ID NO: 3 - protein, 6 aa, synthetic construct
  REGION 1..6: note = (S)-2-amino-N-(17-((5-(dimethylamino)naphthalene)-1 -sulfonamido)-2,5,8,11,14-pentaoxo-3,6,9,12,15-pentaazahep tadecyl)-3-(1H-indol-3-yl)propenamiden
WGGGGG

(19) United States
(12) Patent Application Publication
(10) Pub. No.: US 2025/0257111 A1
(43) Pub. Date: Aug. 14, 2025

(54) Supramolecular Complex of a Biomolecule

(71) Applicant: Cambridge Enterprise Limited, Cambridge, Cambridgeshire (GB)

(72) Inventors: Oren Scherman, Cambridge, Camrbidgeshire (GB); Xiaoyi Chen, Cambridge, Cambridgeshire (GB); Zehuan Huang, Cambridge, Cambridgeshire (GB); Jade McCune, Cambridge, Cambridgeshire (GB)

(21) Appl. No.: 18/858,224

(22) PCT Filed: Apr. 21, 2023
(86) PCT No.: PCT/EP2023/060454

§ 371 Date: Oct. 18, 2024

(30) Foreign Application Priority Data
Apr. 22, 2022 (GB) 2205916.6
Apr. 28, 2022 (GB) 2206222.8

(51) Int. Cl. C07K 14/62; A61K 47/54; C07K 1/113; C07K 1/34

(52) U.S. Cl. CPC C07K 14/62; A61K 47/545; C07K 1/1136; C07K 1/34

(57) ABSTRACT

The invention provides a ternary complex of a cucurbituril host, such as CB[8], with a first aryl guest and second aryl guest, wherein the first aryl guest is a group within a biomolecule, such as insulin, and the first and second aryl guests are different. Also provided is a method of decomplexing the complex, the method comprising the step of treating the complex with a competitor guest, and permitting the competitor guest to displace at least the first aryl guest from the complex.

The project leading to this application has received funding from the European Union's Horizon 2020 research and innovation programme (European Research Council grant agreement No. 726470 and Marie Sklodowska-Curie grant agreement No. 845640).

### RELATED APPLICATION

The present application claims priority to, and the benefit of, GB 2205916.6 filed on 22 Apr. 2022 (22.04.2022) and GB 2206222.8 filed on 28 Apr. 2022 (28.04.2022), the contents of each of which are hereby incorporated by reference in their entirety.

### FIELD OF THE INVENTION

The invention provides a method for capturing and stabilising a biomolecule, such as a polypeptide, using a cucurbituril supramolecular complex. Also provided is the complex and methods for releasing the biomolecule from the complex on-demand.

### BACKGROUND

Methods for improving the stability, and therefore shelf-life, of biomolecules, such as insulin, are of interest within the medical field.

Webber et al. have explored the modification of insulin with poly(ethylene glycol) (PEG) to achieve an increase in the stability of the polypeptide. Here, insulin is incorporated into a cucurbit[7] uril (CB[7])-PEG conjugate. The CB[7] is covalently connected to the PEG group. The CB[7] forms a binary complex with the phenyl group of the Phe residue at position 1 of the insulin. The host-guest complex is used as an essentially permanent attachment to the insulin, with the conjugate used direct in this form in vivo, and the authors suggest that the presence of the CB complex provides advantages for the in vivo use (such as possible advantages relating to delayed and controlled uptake for higher molecular weight insulins).

Webber et al. show that the cucurbit[7] uril may be used to form complexes with other biomolecules, including glucagon and an anti-CD20 antibody.

However, the modification of a biomolecule is not always beneficial, and a modification may not always maintain, or improve, bioactivity. There is a need for a stabilisation strategy that allows for the recovery and use of the biomolecules in its original form.

### SUMMARY OF THE INVENTION

In a general aspect the invention provides a ternary complex comprising a cucurbituril host holding a first aryl guest and a second aryl guest, where the first guest is a group of a biomolecule, such as a polypeptide.

The second guest differs from the first guest. Thus, the complex is a heterogeneous complex. The formation of the heterogeneous complex may be favoured over the formation of a homogeneous complex, where the guests are identical.

Complexes of this type may be beneficially used to stabilise the biomolecule for storage, and the biomolecule may be easily and rapidly released from the complex when needed. The complex formation thereby limits the degradation of the biomolecule and maintains its activity upon release.

The selective capture of the biomolecule in a complex also allows for the biomolecule to be separated from other components in a mixture, thereby also allowing the at least partial purification of the biomolecules. For example, the complex may be connected to the solid phase allowing the complex to be collected by simple filtration, with separation from other solubilised components of a mixture.

The first aryl guest may be a carboaryl or heteroaryl group present within a side chain of an amino acid, such as an α-amino acid.

The first aryl guest may be a phenyl group, such as present within a phenylalanine residue of a polypeptide. The amino acid residue may be a terminal or mid-chain residue within a polypeptide, and preferably a terminal residue, such as an amino acid residue within the N-terminal region of the polypeptide, such as the N terminal residue.

The polypeptide may be a protein, including an antibody. The protein is preferably insulin, such as human insulin, and including human recombinant insulin. The first guest may be the phenyl group of the phenylalanine residue at position 1 (1-Phe) of insulin, for example within chain B.

The second guest may be a perfluorophenyl group (i.e. pentafluorophenyl). The perfluorophenyl group may be covalently connected directly or indirectly to a solid phase, such as a polymeric solid phase.

In a further aspect of the invention there is provided a method of forming a complex, such as a complex of the first aspect of the invention, the method comprising the step of mixing cucurbituril together with a biomolecule having a first aryl guest, and a second aryl guest, and permitting the host and guests to self-assemble.

The complex may form by self-assembly. The method may be performed, and is preferably performed, under aqueous conditions. The method may be performed, and is preferably performed, at ambient temperature, such as a temperature in the range 5 to 30° C.

In a further aspect there is provided a method of decomplexing a complex of the invention, the method comprising the step of treating the complex with a competitor guest, and permitting the competitor guest to displace at least the first aryl guest, optionally together with the second aryl guest, from the complex.

The method may be performed, and is preferably performed, under aqueous conditions. The method may be performed, and is preferably performed, at ambient temperature, such as a temperature in the range 5 to 30° C.

The method of decomplexing results in releasing a biomolecule from the complex.

The competitor guest may be a compound that is or comprises a competitor guest.

In a preferred embodiment, the competitor compound is memantine (DMADA), which may be used as memantine hydrochloride.

The release of the biomolecules from a complex may include the preliminary step of forming the complex, such as according to the method of the present invention. The release of the complex may be performed 1 week or more after the formation, such as 1 month after the formation. The complex is stable and my prevent degradation of the biomolecule. Thus, the complex may be suitable for long term storage of the biomolecules, which can be released from the complex when needed, and without significant loss of bioactivity.

These and other aspects and embodiment of the invention are described in detail below.

### SUMMARY OF THE FIGURES

FIG. 1 shows a schematic of a) host-enhanced heteropeptide dimer formed from F′GG, WGG, and CB[8]; b) molecular structures of FGG, YGG, NpGG; c) on-resin recognition through interfacial heterodimerization.

FIG. 2 shows a) ¹H & ¹⁹F NMR spectra (D2O, 298 K) obtained through titration of WGG (16.0 mM) into F′GG-CB[8](1.0 mM); b) HR ESI-MS spectra of heteropeptide dimers (H2O, 1.0 mM) of WGG, FGG, YGG, NpGG with 1:1 complex of F′GG-CB[8]; ITC titration plots (10 mM phosphate buffer, pH 7.0, 298 K) of c) F′GG (3.0 mM) into CB[8](0.1 mM); d) WGG, FGG, YGG, NpGG (3.0 mM) into F′GG-CB[8](0.2 mM); and e) schematic of self-sorting mechanism.

FIG. 3 shows a schematic of a) molecular structure of WGGGGG-dansyl, and b) interfacial recognition with F′GGGGG resin (10 mM); confocal fluorescent images of c) F′GGGGG resin, and those added with d) WGG-CB[8](10 mM), e) WGGGGG-dansyl (10 mM), and f) WGGGGG-dansyl-CB[8](10 mM) obtained through laser excitation at =405 nm under a gray field. Scale bar=200 m. Fluorescence intensity is quantified by mean gray value (MGV).

FIG. 4 shows a) Schematic of on-resin recognition and separation of an aromatic tripeptide from a peptide mixture; b) UV spectra of WGG-CB[8](1.0 mM) before and after treatment with F′GGGGG-resin (10.0 mM); histograms of c) recycling efficiency of WGG in continuous on-resin recognition cycles; d) percentage of WGG obtained after multi-cycle isolation; e) schematic of on-resin stabilization of insulin, on-demand release and binding to CB[8], not drawn to scale; f) UV spectra of insulin (0.2 mM) before and after treatment with F′GGGGG-CB[8]-resin (10.0 mM) followed by release at days 0, 2, 5, & 8; g) histograms of recycling efficiency of insulin at days 0, 2, 5, & 8.

FIG. 5 shows UV spectra of control experiments of insulin (0.2 mM) on-resin recognition. a) insulin (0.2 mM) on-resin recognition on F′GGGGG-resins (10 mM); b) insulin (0.2 mM) with CB[8](1.0 mM) on-resin recognition on GGGGGG-resins (10 mM); c) insulin (0.2 mM) on-resin recognition on GGGGGG-resins (10 mM); d) insulin (0.2 mM) with CB[8](1.0 mM) on-resin recognition on blank resins (H-Rink Amide ChemMatrix resin) (phosphate buffer, H₂O).

FIG. 6 shows UV spectra of insulin (0.2 mM) solutions before and after treatment with F′GGGGG-CB[8]-resin (10.0 mM) followed by time-dependent release at days 0, 2, 5, and 8 (phosphate buffer, H₂O).

FIG. 7 shows CD spectra of insulin solutions before and after treatment with F′GGGGG-CB[8]-resin followed by time-dependent release at day 2, 5, & 8 (phosphate buffer, H₂O).

FIG. 8 shows ¹H NMR of on-resin selective isolation of aromatic peptides from mixed peptide mixture (WGG, KGG, EGG, LGG 1.0 mM, phosphate buffer, D₂O) over 3 cycles using F′GGGGG-resins (10 mM).

### DETAILED DESCRIPTION OF THE INVENTION

The present inventors have found that a CB[8] host may be used to form a complex together with a biomolecule, such as a polypeptide, and a second component, which is selected for the formation of a heterogenous pairing with a group of the biomolecules in the host cavity.

The complex has been found to stabilise a biomolecule, such as insulin, for storage, and the biomolecule may be released on demand for use, and without loss of biomolecule integrity, including structural and functional integrity.

Li et al. describe the use of a CB[7] to form a binary complex with insulin or human growth hormone (hGH). Here, the CB[7] is covalently connected to the solid phase. The insulin and HGA are releasable from the complex upon treatment with a competitor guest. However, Li et al. do not show or suggest that the formation of the complex is associated with a stabilisation of the insulin or hGH biomolecule.

Also, the preparation of CB[7], and its covalent connection to the solid-phase, is not straightforward, and is onerous. Thus, accessibility and scalability are a problem. In contrast, the use of readily available and easily prepared CB[8], and related larger cucurbituril hosts, as described in the present case, avoids the problems associated with the use of CB[7] and its derivatives.

### Complex

A complex of the invention comprises a cucurbituril host, such as CB[8], holding non-covalently within its cavity a first guest and a second guest. This is supramolecular complex, and it is also a heterogenous complex, and the first and second guests differ.

The cucurbituril forms a non-covalent bond to each guest. The present inventors have previously established that complexes of cucurbituril are readily formed and can be used to provide robust non-covalent connections between compounds.

A complex may be formed between the cucurbituril and a first guest from a biomolecule, and the formation of the complex is tolerant of many functionalities within the monomer.

The complex also comprises the second guest, and in the preferred embodiments of the invention, this second guest is connected to a solid phase. The formation of the complex is also tolerant of such solid phase functionality.

Where the complex comprises two guests within the cucurbituril cavity, the association constant, Kₐ, for that complex is at least 10³ M⁻², at least 10⁴ M⁻², at least 10⁵ M⁻², at least 10⁶ M⁻², at least 10⁷ M⁻², at least 10⁸ M⁻², at least 10⁹ M⁻², at least 10¹⁰ M², at least 10¹¹ M⁻², or at least 10¹² M⁻². Preferably the association constant is at least 10¹⁰ M⁻².

The cucurbituril hosts two guest molecules, and the guest molecules are different. The formation of a ternary host-guest complex is believed to proceed via an intermediate binary complex.

The references in the present case to first and second guests is not a description of the order in which the guests complex with the cucurbituril host. Thus, the intermediate binary may form with the second guest and the host, or the first guest and the host.

The formation of the complex is reversible. The separation of a guest from the cucurbituril host, may be referred to as decomplexation.

The decomplexation of the complex to separate the host and the guests may occur in response to an external stimulus, including, for example, a competitor guest compound, light, an oxidising or reducing agent, electrochemical potential, and temperature changes amongst others. In the methods of the present invention, the decomplexation of the complex is undertaken under conditions that will not damage or interfere with the structure of the biomolecule. For this reason, displacement methods involving a benign competitor guest compound are favoured.

A competitive guest for use in the decomplexation of a CB[8]-based network is or comprises adamantane amine (ADA) or memantine (DMADA). A competitor guest may be used in excess to the amount (mole amount) of guests present in the complex. In one embodiment, the competitive guest has a higher association constant than a guest of the complex.

A decomplexation reaction is reversible. Thus, a complex may be permitted to reform, if needed, after the removal of the external stimulus, including for example the removal of a competitor guest compound.

### Cucurbituril and CB[8]

The complex of the invention has a cucurbituril host. The cucurbituril is a cucurbituril capable of hosting two guests. Thus, the host may be a CB[8] host, including variant and derivative forms.

Cucurbiturils that are capable of forming ternary complexes and binary complexes find use in the present invention. There are many examples in the art of cucurbiturils for use in forming such complexes.

Recent work has shown that cucurbituril compounds have high in vitro and in vivo biocompatibility and have extremely low toxicity (see Uzunova et al. Org. Biomol. Chem. 2010, 8, 2037-2042). Thus, when used together with non-toxic polymer components, the present hydrogels are also suitable for use in biological systems.

The cucurbituril should be capable of forming a ternary complex. For example, CB[8], is capable of forming a ternary complex. As too are CB[10] and CB[12] compounds.

The cucurbituril may be a CB[8], CB[10] or CB[12] compound.

In one embodiment, the cucurbituril is a CB[8] compound.

References to a cucurbituril compound are references to variants and derivatives thereof.

A variant of CB[8] may include a structure having one or more repeat units that are structurally analogous to glycoluril. The repeat unit may include an ethylurea unit. Where all the units are ethylurea units, the variant is a hemicucurbituril. The variant may be a hemicucurbit[12] uril (shown below, see also Lagona et al. Angew. Chem. Int. Ed. 2005, 44, 4844).

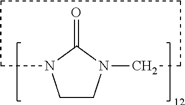

Cucurbituril derivatives are provided and find use in the methods described herein. A derivative of a cucurbituril is a structure having one, two, three, four or more substituted glycoluril units. A substituted cucurbituril compound may be represented by the structure below:

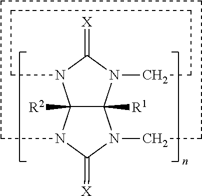

wherein:
n is an integer of at least 8;
and for each glycoluril unit
   each X is O, S or NR³, and
   —R¹ and —R² are each independently selected from —H and the following optionally substituted groups: —R³, —OH, —OR³, —COOH, —COOR³, —NH₂, —NHR³ and —N(R³)₂ where —R³ is independently selected from C₁₋₂₀alkyl, C₅₋₂₀carboaryl, and C₅₋₂₀heteroaryl, or where —R¹ and/or —R² is —N(R³)₂, both —R³ together form a C₅₋₂₀heterocyclic ring; or together —R¹ and —R² are C₄₋₆alkylene forming a C₆₋₈carbocyclic ring together with the uracil frame.

In one embodiment, one of the glycoluril units is a substituted glycoluril unit. Thus, —R¹ and —R² are each independently —H for n−1 of the glycoluril units.

In one embodiment, n is 8, 9, 10, 11 or 12.

In one embodiment, n is 8, 10 or 12.

In one embodiment, n is 8.

In one embodiment, each X is O.

In one embodiment, each X is S.

In one embodiment, R¹ and R² are each independently H.

In one embodiment, for each unit one of R¹ and R² is H and the other is independently selected from —H and the following optionally substituted groups: —R³, —OH, —OR³, —COOH, —COOR³, —NH₂, —NHR³ and —N(R³)₂. In one embodiment, for one glycoluril unit, one of R¹ and R² is H and the other is independently selected from —H and the following optionally substituted groups: —R³, —OH, —OR³, —COOH, —COOR³, —NH₂, —NHR³ and —N(R³)₂. In this embodiment, the remaining glycoluril units are such that R¹ and R² are each independently H.

Preferably —R³ is C₁₋₂₀alkyl, most preferably C₁₋₆ alkyl. The C₁₋₂₀alkyl group may be linear and/or saturated. Each group —R³ may be independently unsubstituted or substituted.

Preferred substituents are selected from: —R⁴, —OH, —OR⁴, —SH, —SR⁴, —COOH, —COOR⁴, —NH₂, —NHR⁴ and —N(R⁴)₂, wherein —R⁴ is selected from C₁₋₂₀ alkyl, C₆₋₂₀ carboaryl, and C₅₋₂₀heteroaryl. The substituents may be independently selected from —COOH and —COOR⁴.

In some embodiments, —R⁴ is not the same as —R³. In some embodiments, —R⁴ is preferably unsubstituted.

Where —R¹ and/or —R² is —OR³, —NHR³ or —N(R³)₂, then —R³ is preferably C₁₋₆alkyl. In some embodiments, —R³ is substituted with a substituent —OR⁴, —NHR⁴ or —N(R⁴)₂. Each —R⁴ is C₁₋₆ alkyl and is itself preferably substituted.

### Cucurbituril Guest

As noted above, a guest is a compound that is capable of forming a host-guest complex with a cucurbituril. The term complexation therefore refers to the establishment of the host-guest complex. The host-guest complex is a ternary complex comprising the cucurbituril host and a first guest and a second guest. A first guest is provided as a group on a biomolecule. A second guest is provided as a group on a second component, which component is optionally a solid phase.

Typically such complexes are based around CB[8] and variants and derivatives thereof. The first guest and the second guest are not the same.

In principle, any compound having a suitable binding affinity (such as those mentioned in the discussion of the complex above) may be used in the present invention. The compound used may be selected based on the size of the moieties that are thought to interact with the cavity of the cucurbituril. The size of these moieties may be sufficiently large to permit complexation only with larger cucurbituril forms.

In the present case, each of the first guest and the second guest is an aryl group.

The aryl group may be a carboaryl group or a heteroaryl group, and is preferably a carboaryl group.

The aryl group may be a monocyclic, bicyclic or tricyclic aryl group.

The aryl group may be a monocyclic or bicyclic aryl group.

The aryl group may be unsubstituted or substituted, such as monosubstituted.

Examples of the carboaryl group include phenyl, naphthyl and anthracenyl.

Examples of the heteroaryl group include indolyl.

The first and the second guests are not the same. In some embodiments, the aryl ring may be the same, but the guests differ in the number and/or identity of the substituents to the ring. For example in the worked examples of the present case, a phenyl first guest is used with a second guest that has a phenyl ring, but this ring is perfluorinated.

Each aryl group substituent may be selected from the group consisting of alkyl, halo, —OH, —OR¹, —NH₂, —NHR¹, —NR¹R², where each —R¹ and —R² is independently alkyl.

Alternatively, each aryl group substituent may be selected from the group consisting of alkyl, halo, —OH, —OR¹, —CN, —NO₂, —NH₂, —NHR¹, —NR¹R², where each —R¹ and —R² is independently alkyl.

An alkyl group may be C₁₋₆ alkyl, such as C₁₋₄ alkyl, such as methyl or ethyl, such as methyl.

A halo group may be selected from —F, —Cl, —Br and —I, and is preferably —F.

Typically, one aryl guest is halogenated, such as fluorinated, whilst the other aryl guest is not. It is preferred that the second aryl guest is halogenated, such as fluorinated. Here, it is also preferred that the first aryl guest is not halogenated, for example it is not fluorinated.

The inventors have found that an electron-poor guest, such as a halogenated aryl guest, such as perfluorphenyl, advantageously forms heterocomplexes with an electron-rich guest, such as a substituted aryl guest.

The first guest is typically an aryl group that is unsubstituted or monosubstituted.

The second guest is typically an aryl group that is substituted.

The first guest may be selected from the group consisting of phenyl, naphthyl, such as naphth-2-yl, hydroxyphenyl, such as 4-hydroxyphenyl, and indolyl, such as indol-3-yl.

The first aryl guest may be covalently connected to a methylene group.

The first guest may be phenyl or indolyl, including indol-3-yl.

It is preferred that the first guest is phenyl.

In another embodiment, the first guest is indolyl, including indol-3-yl.

The first guest may be present within a side chain of an amino acid residue.

Where the first guest is phenyl, the amino acid residue may be phenylalanine.

Where the first guest is hydroxyphenyl, an example amino acid residue where the guest is 4-hydroxyphenyl is tyrosine.

Where the first guest is naphthyl, an example amino acid residue where the guest is naphth-2-yl is (2-naphthyl)alanine.

Where the first guest is indolyl, an example amino acid residue where the guest is indol-3-yl is tryptophan.

The first aryl guest may be provided within the side chain of a phenylalanine, tyrosine, indole or naphthylalanine residue.

The second aryl guest may be selected from substituted phenyl, indolyl, such as indol-3-yl, naphthyl, such as naphth-2-yl, and anthracenyl, such as anthracene-2-yl. Here, the aryl group is substituted with one or more substituents. The aryl group may be per-substituted.

That is, each available ring position may be substituted. Here, each substituent may be a halo, and preferably each substituent is fluoro.

The second aryl guest may be covalently connected to a methylene group.

The second aryl guest may be phenyl having five halo substituents, such as five fluoro substituents. Thus, the second aryl guest may be perfluorophenyl (pentafluorphenyl), and this is preferably connected to a methylene group.

The second aryl guest may be indolyl, such as indol-3-yl, having from 1 to 5 halo substituents, such as 1 to 5 fluoro substituents.

The second aryl guest may be naphthyl, such as naphth-2-yl, having from 1 to 7 halo substituents, such as 1 to 7 fluoro substituents.

The second aryl guest may be anthracenyl, such as anthracene-2-yl, having from 1 to 9 halo substituents, such as 1 to 9 fluoro substituents.

The second aryl guest may be present within a side chain of an amino acid residue. Where the second guest is perfluorophenyl, the amino acid residue may be perfluorophenylalanine.

An amino acid residue containing the second aryl guest may be provided within a polypeptide sequence having two or more contiguous amino acid residues, such as three or more, such as 4 or more amino acid residues, such as 5 or more, such as 6 or more amino acid residues.

The amino acid residue having the second aryl guest may be provided at the N terminal of the polypeptide sequence. The terminal amino acid residue may be connected to a polypeptide sequence having one or more, such as three or more, such as 4 or more, such as 5 or more amino acid residues that are contiguous with the terminal amino acid residue.

Preferably, the polypeptide sequence has an amino acid residue with the second aryl guest at the N terminal, and this is connected to one or more, such as three or more, such as 4 or more, such as 5 or more glycine residues that are contiguous with the terminal amino acid residue.

The second aryl guest may be covalently linked, directly or indirectly, to a solid phase.

The solid phase may be a polymeric phase, such a polystyrene resin or poly(ethylene glycol) (PEG) resin, such as a Rink Amide ChemMatrix Resin. Here, the formation of the complex connects the biomolecule to the solid-phase via the non-covalent interaction in the complex.

Once the biomolecule is immobilised, it may be separated from other components, which are not insolubilized, by simple filtration. Subsequent release of the biomolecule from the complex, for example using the decomplexation methods described herein, then provides the biomolecules in a higher purity form.

The aryl guest may be connected to an optionally substituted imidazolium group, such as connected to the optionally substituted imidazolium group through methylene. The aryl guest may be connected, such as through methylene, to the 1-position of the imidazolium ring nitrogen.

The 2-, 4- and 5-positions of the imidazolium group are preferably unsubstituted. The 3-position of the imidazolium group is optionally substituted and may be substituted.

The imidazolium 3-position may be substituted with alkyl, such as C₁₋₄ alkyl, such as methyl or ethyl, such as methyl.

The imidazolium 3-position may be connected to a methylene group.

The imidazole group may be covalently linked, directly or indirectly, to a solid phase. The connection to the solid phase may be made via the imidazolium 3-position, such as through a methylene group connected to the imidazolium 3-position.

An amino acid residue as described herein may be an α-amino acid residue, a β-amino acid residue or a γ-amino acid residue.

An amino acid residue as described herein may be an α-amino acid residue.

The amino acid residue may be an L- or D-form, and is typically L-form.

In a preferred embodiment, both the first guest and the second guest are groups within the side-chain of an amino acid residue. Preferably, each amino acid residue has the same stereoform, such as both amino acid residues having the L-form.

### Biomolecule

The biomolecule may be selected from a polypeptide, a polynucleotide and a polysaccharide.

The present inventors have found that the ternary complex of the invention stabilised a biomolecule, such as insulin, preventing its degradation. The complex may be stored without appreciable loss of structural or functional activity.

The biomolecule may inherently contain a group that is suitable for use as a guest in a ternary complex of the invention. For example, a polypeptide may contain a phenylalanine residue, and the phenyl group in the side chain of that residue may be a suitable guest.

In other embodiments a biomolecule may be modified to include a guest aryl group so that the biomolecule may be held in complex.

A guest group present within a biomolecule is available for interaction with the cucurbituril host. Thus, a first aryl guest may be provided at a terminal of a biomolecule, where it is more likely to be freely available for interaction with a host. However, a guest group may also be provided with mid-chain if that guest is available for interaction. It is preferred that the first aryl guest is provided at a terminal region of a biomolecule.

In one embodiment, the biomolecule has a molecular weight of at least 100, at least 200, at least 300, at least 1,000 (1 k), at least 5,000 (5 k), at least 10,000 (10 k), at least 15,000 (15 k), at least 20,000 (20 k), at least 50,000 (50 k), at least 100,000 (100 k) or at least 200,000 (200 k).

The polypeptide may be a protein, such as a peptide hormone or an antibody.

Thus, the biomolecule may be selected from the group of polypeptides consisting of:
Blood factors such as Factor VIII and Factor IX;
Thrombolytic agents, such as tissue plasminogen activator;
Peptide hormones, such as insulin, glucagon, growth hormone, gonadotrophins;
Haematopoietic growth factors, such as Erythropoietin, colony-stimulating factors;
Interferons, such as Interferons-α, -ρ, -γ;
Interleukin-based products, such as Interleukin-2;
Vaccines, such as Hepatitis B surface antigen;
Antibodies, including Monoclonal antibodies; and
Other proteins such as tumour necrosis factor, and therapeutic enzymes.

Preferably, the biomolecule is a peptide hormone, such as insulin. The polypeptide includes proteins having post-translation modifications. The polypeptide may also include non-natural amino acid residues, and the polypeptide may also include with amino acid residues that are modified during post-translation modification.

A polypeptide may have at least 10 contiguous amino acid residues, such as at least 20, at least 50, at least 100 or at least 500 amino acid residues.

The worked examples in the present case use insulin as an exemplary polypeptide, and connections are formed to the 1-Phe residue within insulin within chain B. Webber et al. have also formed cucurbituril complexes with the 1-Phe residue within insulin, albeit using CB[7] and in binary complex only. Webber et al. also shown that cucurbituril complexation with other biomolecules is possible, such as other polypeptides, including glucagon and an anti-CD20 antibody. A skilled person will appreciate that the present invention may be used to form complexes with biomolecules other than insulin.

The first guest may be provided at the N or C terminal regions of a polypeptide, and preferably the N terminal. Here, a terminal region may be regarded as the three contiguous amino acid residues at the N or C terminal. Typically, the first guest is present within the amino acid residue at the N or C terminal itself, and preferably the N terminal. The inventors have found that the association constant for the first guest may be greatest when the first guest is present with the amino acid residue at the terminal itself, with the association constants decreasing as the first guest is provided at further distance from the N terminal, such as within the second and third amino resides from the N terminal.

Where, the biomolecule is a polypeptide, the first guest is typically present in an arylalanine residue within the polypeptide.

Where, the biomolecule is a polypeptide, the first guest is typically present in a phenylalanine, tyrosine, indole or naphthylalanine residue within the polypeptide.

A polynucleotide may be a DNA, RNA or a related from such as a PNA, and may be single stranded.

A polynucleotide may have at least 10 contiguous nucleotides, such as at least 50, at least 100 or at least 500 nucleotides.

The first guest may be provided at the 3′ or 5′ terminal region of the polynucleotide.

A polysaccharide may contain two or more, such as three or more contiguous sugar groups.

A biomolecule may a naturally occurring biomolecule or it may be a variant, such as a mutant, of such a biomolecule.

The biomolecule may also be a modified form, for example one that contains a label for detection or capture. Typically, such functionality is provided away from the first guest, so as not to interfere with the formation of the complex.

A biomolecule may also be in protected from, such as chemically protected form.

Typically, the biomolecules is not itself immobilised on a solid phase. Rather, the formation of the complex may serve to non-covalently connect the biomolecule to a solid phase through its interaction with a second guest in the complex, where the second guest is connected to the solid phase, as present in a preferred embodiment of the invention.

A biomolecule may be provided in an aqueous mixture, and the biomolecule may be provided together with other components. Here, the formation of the complex, preferentially comprising the biomolecule over the other components, may serve as a process for purifying the biomolecule from those components.

These other components may not possess a suitable guest group for formation of a complex, or a functional group may be present, but unavailable for formation (for example, because that guest is internalised within the other component).

### Insulin

The present invention is well suited to the formation of complexes where the biomolecule is insulin.

The insulin may be a polypeptide having the amino acid sequence corresponding to human insulin.

The insulin may be an insulin that is recombinantly produced. Thus, the insulin may be human recombinant insulin.

In a preferred embodiment, the first guest is the phenyl group present in the phenylalanine residue present at position 1 of insulin, particularly human insulin.

The insulin need not be provided in sustainably purified form. The methods of the invention may allow for the preferential formation of the complex with insulin over other compounds present. Once formed, the complex may be readily purified from other components, for example by simple filtration where the complex includes a solid-phase bound component, such as the second compound.

### Formation and Decomplexation of a Complex

The invention provides a method of preparing a complex of the invention, the method comprising the step of mixing a cucurbituril host together with a biomolecule having a first aryl guest, and a second aryl guest, and permitting the host and guests to self-assemble.

The method may further comprise the step of at least partially purifying the complex. For example, the complex may be a least partially separate from one or more other components that are not in the complex.

It is preferred that the second aryl guest is bound to the solid phase, and it follows that the resulting complex is also bound to the solid phase. Here the complex may be at least partially purified by collection of the solid phase, for example by filtration, to separate the complex from other components.

The invention also provides a method of decomplexing a complex of the invention, the method comprising the step of treating the complex an external stimulus, and permitting the external stimulus to displace at least the first aryl guest from the complex.

The release of the biomolecule may be achieved as needed, and may be regarded as on-demand release.

The external stimulus may the addition of a competitor guest. The competitor guest may be or comprise adamantane amine (ADA) or memantine (DMADA).

The formation and the decomplexation may be performed under aqueous conditions.

Adventurously, the formation and decomplexation steps may also be performed at ambient temperatures, such as a temperature in the range 5 to 30° C.

The complexation and decomplexation may be performed at temperatures that are not associated with a degradation of the biomolecule, and the integrity of the biomolecule may be preserved in the complexation and decomplexation processes.

### Stabilization of a Biomolecule

The complexes of the invention may be used to stabilise a biomolecule for storage, and the biomolecule may be easily and rapidly released from the complex when needed. The complex formation thereby limits the degradation of the biomolecule and maintains its activity upon release.

Here, the complexes of the invention are preferably bound to the solid phase. For example, the second guest is connected to the solid phase, such as a polymeric phase.

Accordingly, there is provided the use of a complex to stabilise a biomolecule, such as a protein.

The stability of a biomolecule may be assessed by structural analysis, such as circular dichroism, and may include a comparison of the secondary and tertiary structures of the biomolecules before and after complexation. The satiability of a biomolecule may be assessed by a suitable activity assay, which may include cells-based studies.

### Purification of a Biomolecule

The selective capture of the biomolecule in a complex also allows for the biomolecule to be separated from other components in a mixture, thereby also allowing the at least partial purification of the biomolecules. For example, the complex may be connected to the solid phase allowing the complex to be collected by simple filtration, with separation from other solubilised components of a mixture.

Accordingly, there is provided a method for at least partially purifying a biomolecule from a mixture, the method comprising the step of providing a biomolecule in a mixture, such as an aqueous mixture, where the biomolecule has a first aryl guest; providing a cucurbituril host and a second aryl guest in the mixture; permitting the host and guests to self-assemble; and subsequently separating the resulting complex from the mixture.

### Experimental

On account of their high binding affinity and range of guests, cucurbit[n] uril (CB[n]) macrocyclic hosts are ideal to bind peptides⁶⁻¹¹ Urbach and co-workers reported a homopeptide dimer between two FGG tripeptides and CB[8], displaying high binding strength (K ca. 10¹¹ M⁻²)⁶. This homodimer has been adopted as a versatile building block in the design and fabrication of supramolecular oligomers,¹² polymers,^{13,14} hydrogels,^{15,16} and protein/peptide assemblies.¹⁷ Although significant advances have been made, a major challenge remains favorable, quantitative formation of heteropeptide dimers without homodimerization.

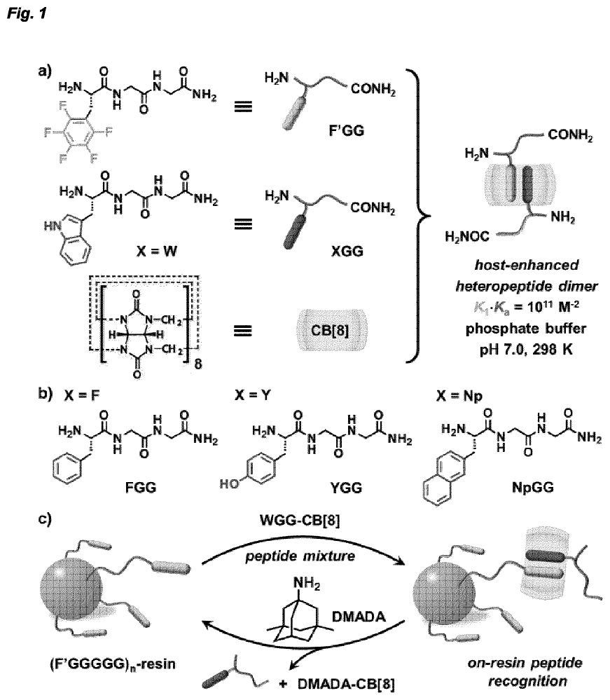

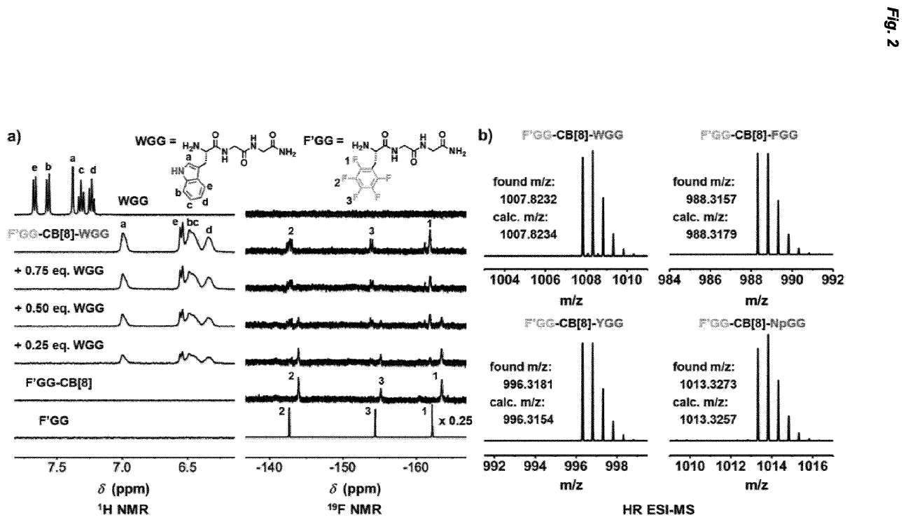

Herein, we employ CB[8] to mediate heterodimerization of a canonical aromatic peptide and a non-canonical L-perfluorophenylalanine (F′)-containing peptide, FIG. 1a. Recently, we reported that an electron-poor perfluorophenyl first guest and an electron-rich phenyl second guest can exclusively form a CB[8]-mediated heteroternary complex through host-enhanced polar-π interactions.^{18,19} Thus, we postulated that the F′-containing peptide (F′GG) would exclusively form a 1:1 complex with CB[8], avoiding homodimerization on account of the electrostatic repulsion within a 2:1 complex. Subsequent association of various aromatic peptides (e.g. WGG) with the F′GG-CB[8] complex may enable access to a new host-enhanced heteropeptide dimer with superior binding strength, FIG. 1a.

Five model aromatic tripeptides containing Lperfluorophenylalanine20 (F′GG), L-tryptophan (WGG), L-phenylalanine (FGG), L-tyrosine (YGG), and L-(2-naphthyl)alanine (NpGG) at the N-termini were designed and prepared, FIG. 1a-b. An equimolar mixture of F′GG, CB[8], and XGG should result in an exclusive heterodimer instead of an equilibrium mixture containing homodimers. Two additional series of tripeptides (GGX, GXG), containing aromatic amino acids either at the C-termini or in the mid-chain, were synthesized to investigate a range of second guests and the effect of their position in the oligopeptides (shown below). After elucidating binding thermodynamics, we applied this heterodimerization to achieve on-resin recognition and isolation^{21,22} of aromatic tripeptides from a peptide mixture exhibiting high efficiency and selectivity, FIG. 1c.

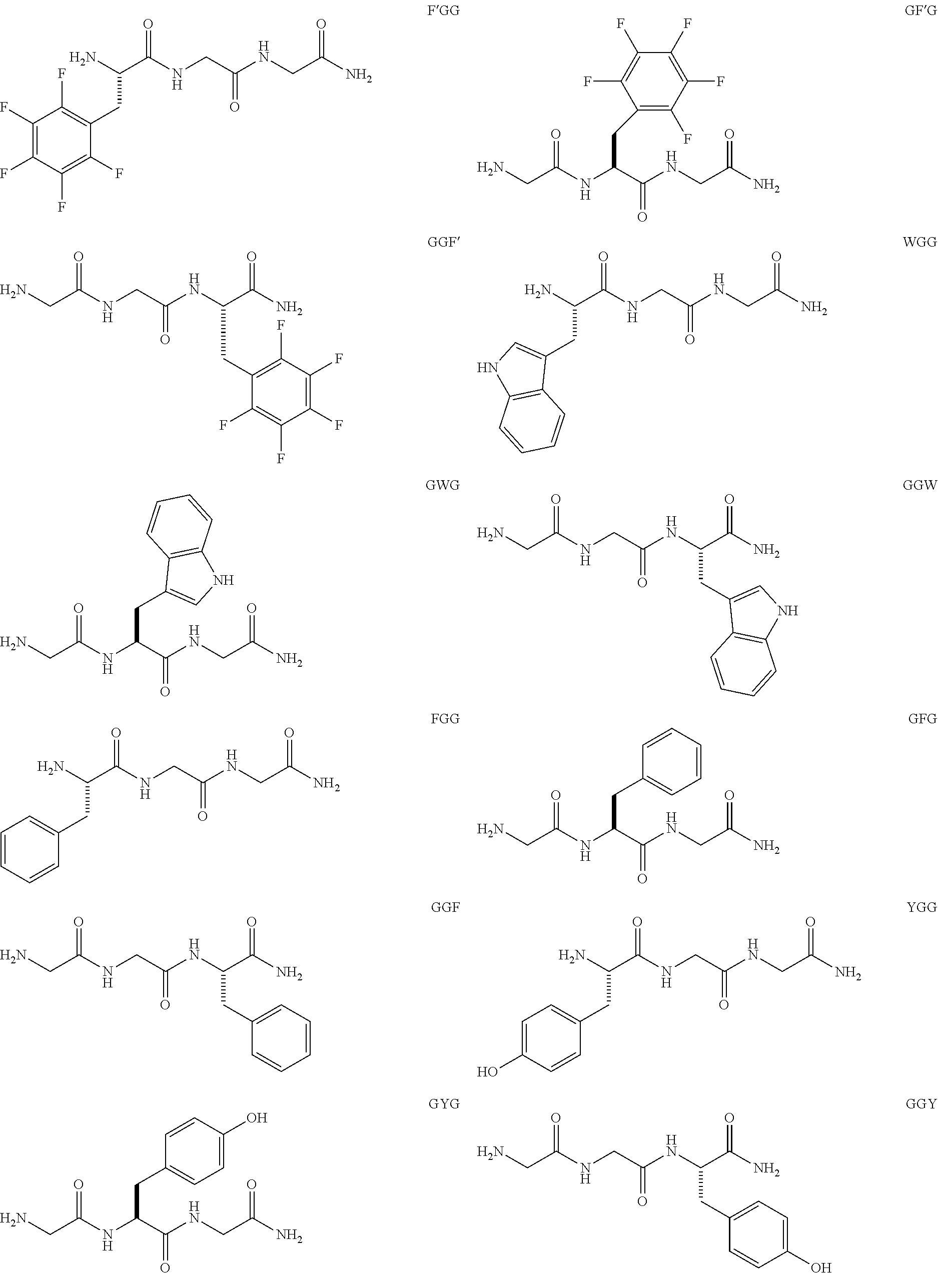

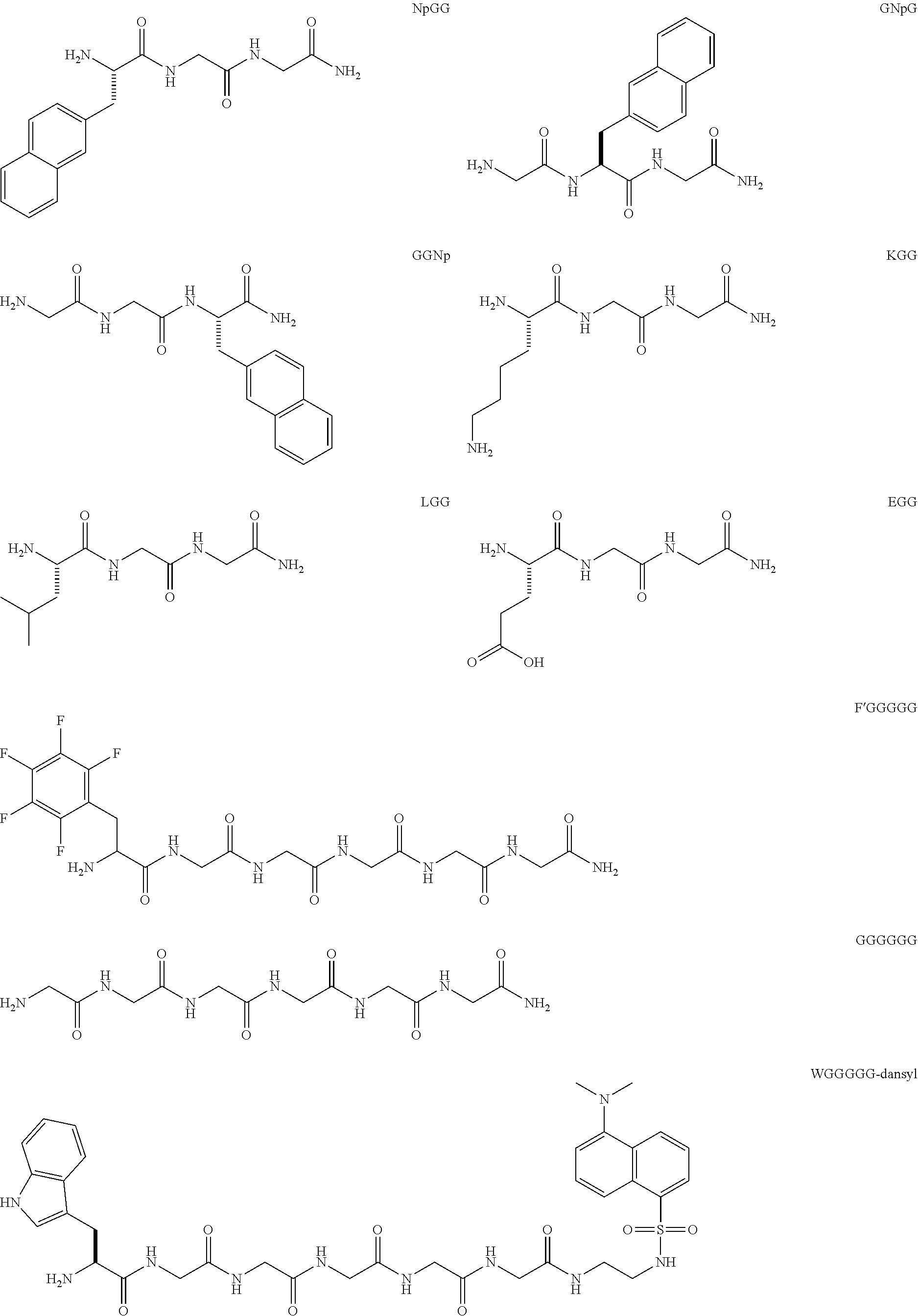

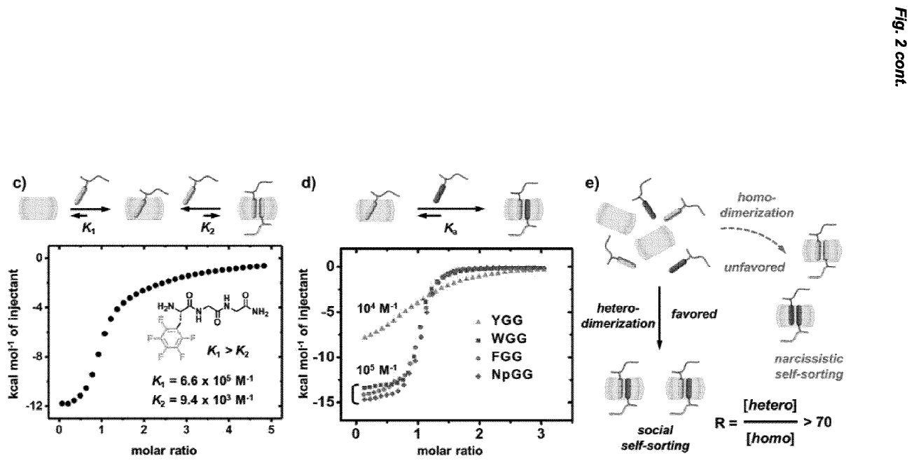

¹H and ¹⁹F NMR titrations were performed to probe heteropeptide dimerization within CB[8], FIG. 2a (and additional data not shown). Titration of WGG into a 1:1 mixture of F′GG-CB[8] resulted in a gradual appearance of indole protons at 6.25-7.10 ppm. On account of shielding from the CB[8] cavity, these proton peaks exhibited upfield shifts compared to free WGG, suggesting that the indole group of F′GG-CB[8]—WGG is located in a different chemical environment from unbound WGG. This titration was also monitored by ¹⁹F NMR, FIG. 2a; a new group of fluorine peaks gradually appeared, while the peaks of F′GG-CB[8] disappeared. Additionally, equivalent mixtures of F′GG, CB[8], and XGG were characterized by high-resolution ESI-MS, FIG. 2b. All ion peaks for F′GG-CB[8]-WGG, F′GG-CB[8]-FGG, F′GG-CB[8]-YGG and F′GG-CB[8]-NpGG complexes were identified at their calculated m/z values. Together, these data confirmed the successful formation of new heteropeptide dimers.

Isothermal titration calorimetry (ITC) was employed to study binding thermodynamics of heteropeptide dimerization (FIG. 2c and additional data not shown). Titration of F′GG (3.0 mM) into CB[8](0.1 mM) led to a stepwise binding curve with two transitions at molar ratios of 1.0 and 2.0 (FIG. 2c). The first-step binding constant (K₁) is 6.6×10⁵ M⁻¹, while the second-step (K₂) is 9.4×10³ M⁻¹. This indicates negative cooperativity where K₁>K₂^{18,19,23} Such favourable 1:1 complexation enables secondary access of electron-rich aromatic peptides. Titrations of XGG (X=W, F, Y, Np; 3.0 mM) into F′GG-CB[8](0.2 mM) resulted in four binding curves with a clear transition at 1.0 molar ratio (FIG. 2d), indicating successful incorporation of XGG into F′GG-CB[8].

Table 1 shows that all XGG peptides exhibited high binding strengths (Kₐ>10⁴ M⁻¹), confirming thermodynamic stability of heteropeptide dimerization. YGG (FIG. 2d, green) displayed a relatively low Kₐ as the para-substituted hydroxyl group may decrease enthalpic contributions, weakening the second association.²⁴ Nevertheless, the overall binding constants (K1 Kₐ) for the heteropeptide dimers F′GG-CB[8]-XGG are all higher than 10¹⁰ M⁻². This shows significant enhancement compared to their parent dimers (e.g. F′GG-WGG,^{25,26} K_{dimer} 1 M⁻¹). The overall K₁ Kₐ for F′GG-CB[8]-FGG (heterodimer 2.4×10¹¹ M⁻²) is higher than 2FGG-CB[8](homodimer, 1.5×10¹¹ M⁻²),⁶ on account of the enhanced polar-interactions. Notably, no secondary association of non-aromatic analogs (KGG, EGG, LGG) was observed, highlighting selectivity for aromatic over non-aromatic peptides.

To understand influence of aromatic position on heterodimerization, Kₐ of GXG and GGX (X=W, F, Y, Np) with F′GG-CB[8] were determined by ITC. Shifting aromatic residues from N to C-termini led to a notable decrease in Kₐ, Table 1. The increased distance between the positive charge at the N-terminus and the aromatic motif weakens ion-dipole interactions at the CB[8] portal, reducing the secondary binding affinity. This is exemplified by GGY, where no secondary binding to F′GG-CB[8] was observed. Seven new, derivatized heteropeptide dimers, F′GG-CB[8]-GXG and F′GG-CB[8]-GGX, expand the scope of host-enhanced heteropeptide dimerization. The exhibited binding selectivity to aromatic residues is an advantage of this system, enabling access to a range of peptides and proteins. Compared to previous reports on CB[8]-peptide heteroternary complexes,²⁷ the system described here is simply based upon a F′ amino acid, easily accessible for ligation in chemical biology and biochemistry.

TABLE 1
Thermodynamic data for secondary association of aromatic
residue-containing tripeptides and control tripeptides
with the 1:1 complex of F′GG-CB[8]^{a}

| model | Kₐ | ΔHₐ | −TΔSₐ |
|---|---|---|---|
| peptide | (10³M⁻¹) | (kcal mol⁻¹) | (kcal mol⁻¹) |
| WGG | 460 ± 53 | −13.3 ± 0.2 | 5.6 ± 0.2 |
| GWG | 103 ± 6 | −11.9 ± 0.1 | 5.0 ± 0.1 |
| GGW | 90 ± 7 | −14.5 ± 0.3 | 7.8 ± 0.4 |
| FGG | 356 ± 26 | −14.4 ± 0.4 | 6.8 ± 0.3 |
| GFG | 58 ± 3 | −9.1 ± 0.4 | 2.6 ± 0.4 |
| GGF | 22 ± 1 | −7.5 ± 0.2 | 1.5 ± 0.2 |
| YGG | 3 ± 5 | −9.6 ± 0.1 | 3.6 ± 0.2 |
| GYG | 4 ± 0.3 | −4.7 ± 0.2 | −0.2 ± 0.3 |
| GGY | —^{b} | —^{b} | —^{b} |
| NpGG | 382 ± 22 | −14.6 ± 0.2 | 7.0 ± 0.2 |
| GNpG | 165 ± 10 | −12.7 ± 0.4 | 5.6 ± 0.4 |
| GGNp | 90 ± 2 | −12.0 ± 0.1 | 5.2 ± 0.1 |
| KGG | —^{b} | —^{b} | —^{b} |
| EGG | —^{b} | —^{b} | —^{b} |
| LGG | —^{b} | —^{b} | —^{b} |

Note:
^{a}Averaged with three replicates.
^{b}Not detected.

The thermodynamic mechanism behind peptide heterodimerization is attributed to social self-sorting, FIG. 2e, consistent with previous reports.^{18,19} Two pathways exist in an equimolar mixture of F′GG, CB[8], and XGG: social self-sorting (heterodimerization) and narcissistic self-sorting (homodimerization). Compared to homodimerization (Table S1), social self-sorting was favored in the presence of a ratio of hetero- and homopeptide dimers (e.g. WGG, R=2291), confirming quantitative heteropeptide dimerization in the complex mixture. Notably, simply mixing two aromatic tripeptides with CB[8] does not lead to quantitative heterodimerization (data not shown).

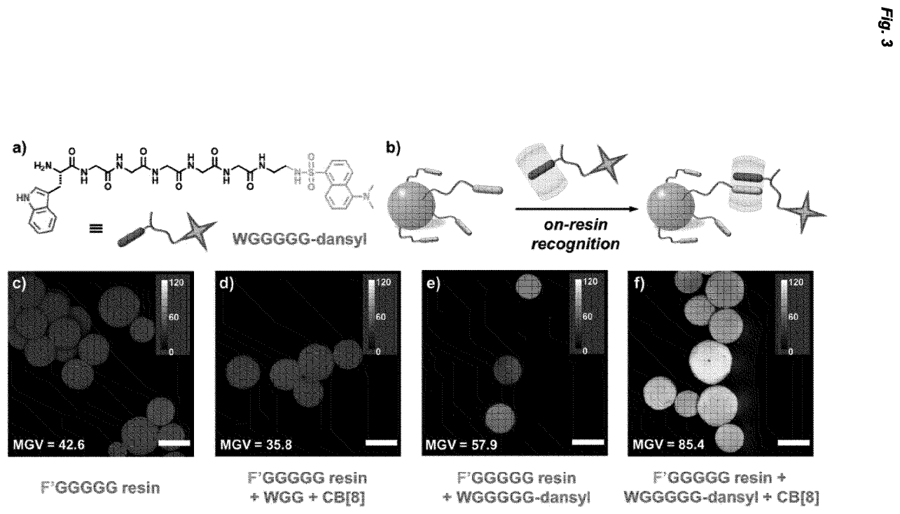

After elucidation of thermodynamics, we demonstrated the utility of heteropeptide dimers to achieve on-resin recognition of aromatic peptides, FIG. 1c. Confocal fluorescence imaging was employed to probe interfacial recognition through a fluorescent tagged peptide (WGGGGG-dansyl),⁹ FIG. 3a-b. A buffered solution of WGGGGG-dansyl-CB[8](10 mM) was mixed with F′GGGGG-functionalized ChemMatrix resin (35-100 mesh particle size, surface loading=0.5 mmol/g) for 10 min at 25° C. with vigorous shaking. Confocal fluorescent images were captured under a gray field upon laser irradiation (λ=405 nm). FIG. 3c-f show that the resin with F′GGGGG-CB[8]—WGGGGG-dansyl displays significantly higher fluorescence than F′GGGGG, F′GGGGG-CB[8]-WGG, and F′GGGGG-WGGGGG-dansyl (data not shown), indicating the successful heteropeptide dimerization on resin.

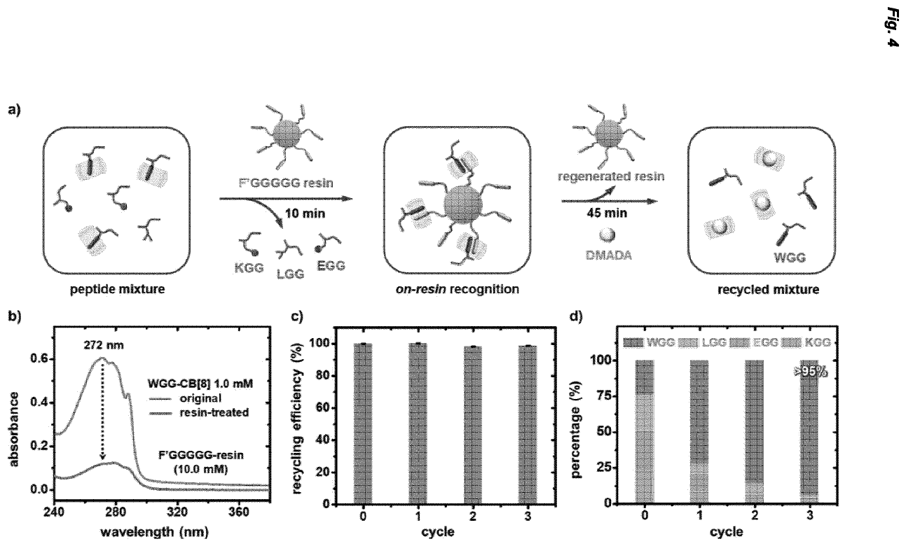

UV experiments were performed to test absorption efficiency through quantification of aromatic peptides present before and after on-resin treatment, FIG. 4a. A typical experiment involved mixing WGG-CB[8](1.0 mM) with F′GGGGG-resin (10.0 mM) at 25° C. for 10 min. The absorption intensity of the resin-treated WGG-CB[8](gray) showed a decrease compared to the original (purple), FIG. 4b. The absorption efficiency for on-resin recognition was 77%, while recognition of free WGG by physical absorption was only 19% (data not shown). Absorbed WGG was released and recycled through competitive binding by memantine hydrochloride (DMADA), regenerating the resin (additional data not shown).

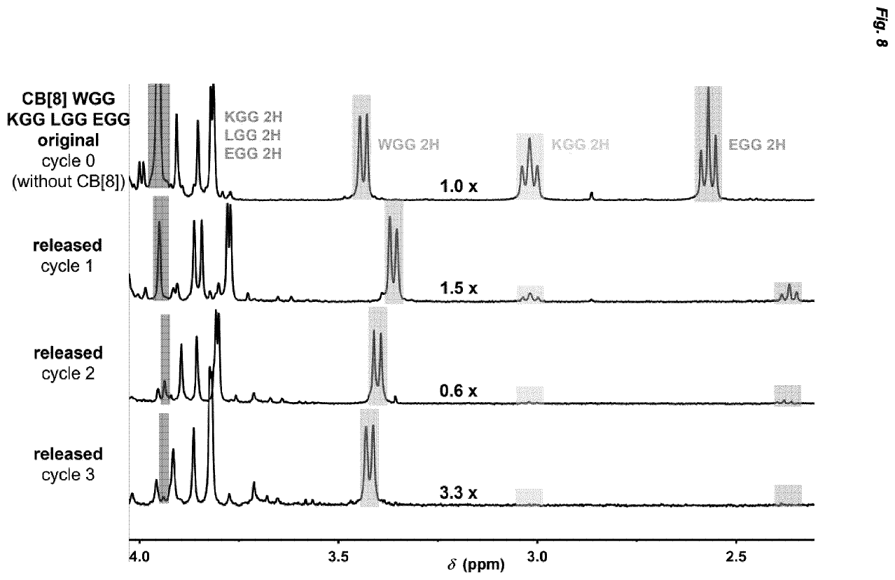

Multi-cycle on-resin recognition was performed to evaluate recyclability, FIG. 4c (and additional data not shown). Recognition regeneration experiments on WGG-CB[8] were repeated for 3 cycles using the same batch of resin. The on-resin recycling efficiency was maintained above 98% over multiple cycles (FIG. 4c), on account of complete release of WGG without any residue accumulation. This confirms regeneration of F′GGGGG-functionalized resin, endowing the whole process with high sustainability for practical use. We further investigated selective isolation of aromatic peptides through recognition-release experiments over 3 cycles (FIG. 4d, and Table 4 and the related discussion below, together with FIG. 8), using a peptide mixture of WGG, KGG, EGG, and LGG ([XGG]=1.0 mM) in the presence of 1.0 mM CB[8]. The ratio of WGG (purple) within the mixture is increased from 26 to >95% after 3 cycles (FIG. 4d). Additionally, residual DMADA and DMADACB[8] can be removed through liquid-phase chromatography. This facile strategy to obtain aromatic peptides with high purity through on-resin heteropeptide dimerization is readily amenable to automation.

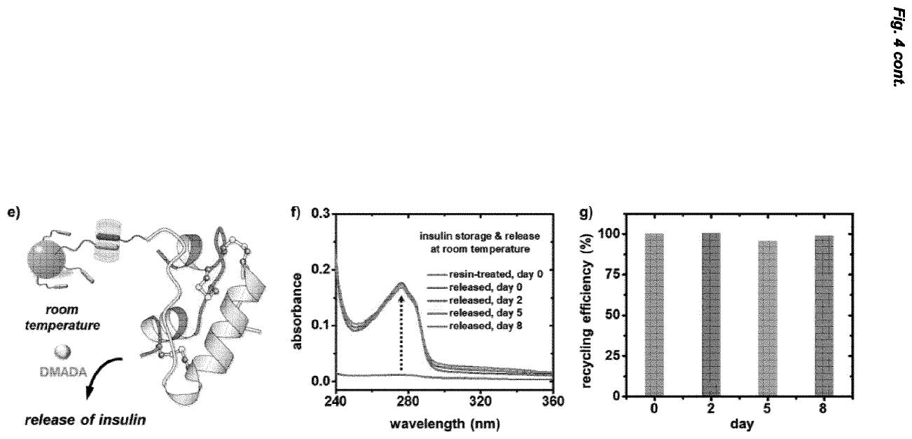

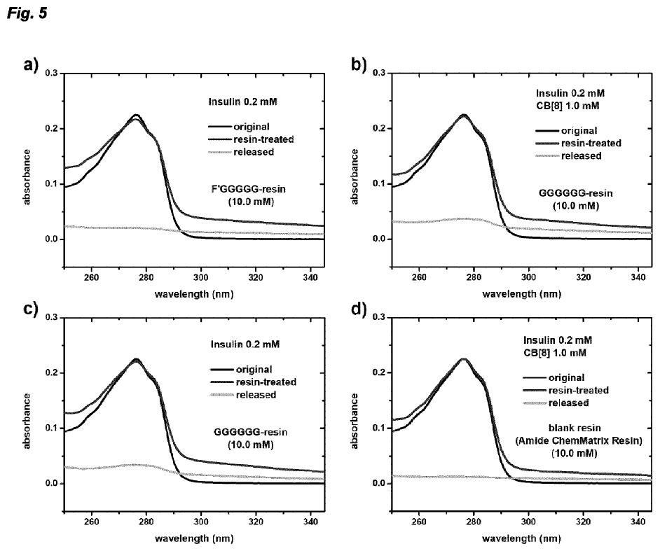

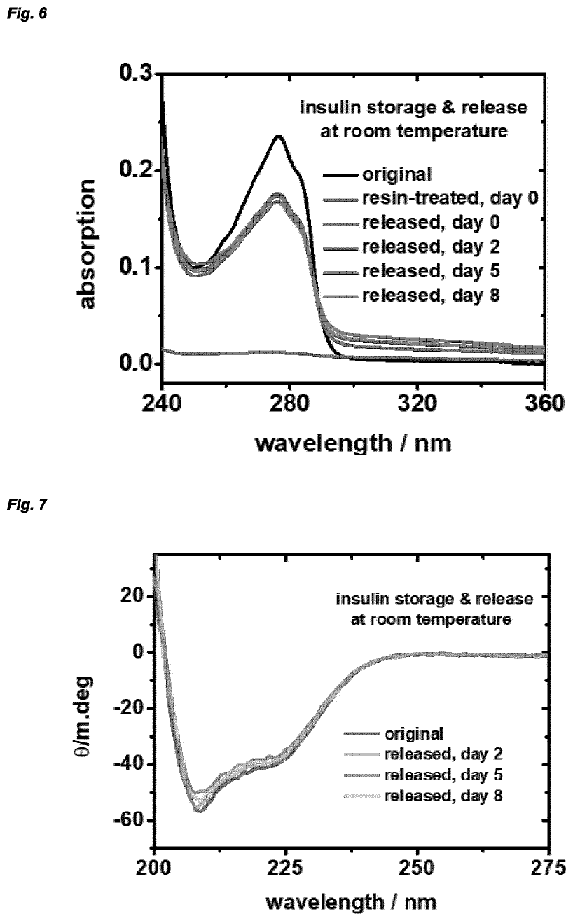

To extend applicability of this approach, we exploited interfacial recognition for insulin stabilization and its on demand release from the resin, FIG. 4e and Table 2 (see later below). Insulin is a widely-used biopharmaceutical for diabetes treatment;²⁸ however, on account of limited stability it requires strict storage conditions (e.g. 2-6° C.) as it's prone to form immunogenic fibrillar aggregates in solution.29 Insulin has a N-terminal phenylalanine, which can serve as a guest UV experiments quantified insulin absorbance onto the resin and on-demand release. Insulin absorption efficiency was calculated to be 94% and its absorption intensity decreased after treatment with F′GGGGG-CB[8] resin, FIG. 4f and FIG. 6 (see also FIG. 5 with UV spectra for insulin on-resin). Through competitive binding, insulin was displaced by DMADA with 95% recycling efficiency (FIG. 4g) over 8 days of storage at room temperature, indicating long-term stability (FIG. 7 and Table 3 as set out below). Our approach provides a route for storing insulin under ambient conditions, removing the current need for refrigeration.

In conclusion, we have introduced a new type of quantitative heteropeptide dimerization. Through host-enhanced polar-interactions, the binding affinity between aryl and perfluorophenyl groups from two different peptides is significantly enhanced with a Kₐ up to 10⁵ M⁻¹ and a K₁ Kₐ up to 101 M⁻², ensuring exclusive formation of heteropeptide dimers. To demonstrate utility, the solution-phase host-guest complex (F′GG-CB[8]-XGG) was transferred to a solid-liquid interface achieving on-resin recognition and isolation of aromatic peptides as well as stabilization and on demand release of insulin under ambient conditions.

This generic approach enables accumulation and separation of aromatic-abundant biomacromolecules useful in biomedical research. We anticipate that this work will inspire research into exploitation of heteropeptide dimerization as a versatile strategy for a wide range of life science applications.

### Materials and Instrumentation

### Materials

Unless noted, all the starting materials were purchased from commercial suppliers and were used without further purification: (L)-Fmoc-Glu(OtBu)—OH, (L)-Fmoc-Gly-OH, (L)-Fmoc-Leu-OH, (L)-Fmoc-Lys(Boc)-OH, (L)-Fmoc-Phe-OH, (L)-Fmoc-Trp(Boc)-OH, (L)-Fmoc-Tyr(tBu)-OH, (L)-Fmoc-3-(2-naphthyl)-alanine-OH, piperidine, diethyl ether, (L)-Fmoc-pentafluorophenylalanine-OH, diisopropylethylamine (DIPEA), trifluoroacetic acid (TFA), triisopropylsilane (TIPS), N,N,N′,N′-tetramethyl-O-(1H-benzotriazol-1-yl)uronium hexafluorophosphate (HBTU), dimethylformamide (DMF), dichloromethane (DCM), monobasic sodium phosphate (NaH₂PO₄), dibasic sodium phosphate (Na₂HPO₄), deuterium oxide (D2O, D, 99.8%), 1,3-dimethylaminoadamantane hydrochloride (memantine hydrochloride, DMADA), Rink Amide-AM Resin, H-Rink Amide ChemMatrix Resin (surface loading of functionalization ca. 0.5 mmol/g), Insulin (Human Recombinant). Phosphate buffer solution was prepared by adding 10 mM NaH₂PO₄ solution to 10 mM Na₂HPO₄ solution until pH equal to 7.0. Cucurbit[8] uril (CB[8]) was synthesized and purified according to a previous report (see Kim), and the molecular weight for CB[8] was calibrated as 1600 g/mol. Water was obtained from a Milli-Q Integral Water Purification System (22.5 MΩ·cm).

### Peptide Synthesis and Purification

All the tripeptides (XGG, GXG, GGX) were synthesized on an automated microwave peptide synthesizer (Liberty Blue, CEM) using standard 9-fluorenylmethoxycarbonyl (Fmoc) chemistry. Peptides were cleaved from the Rink Amide-AM resin with a mixture of 95% TFA, 2.5% TIPS, and 2.5% H2O and shaken for 1 hr at room temperature. The resin was filtered and washed with the cleavage mixture. After evaporating most of the cleavage mixture, the peptides were crushed out and washed three times with cold diethyl ether. The crude peptides were purified using reverse-phase high-performance liquid chromatography (RP-HPLC) on a Phenomenex C18 Kinetic-Evo column with 5 μm pore size, 110 Å particle size and 150×21.2 mm dimensions. The mobile phase for water contained 0.1% TFA. The purification method gradient was set from 5% acetonitrile and 95% water to 100% acetonitrile. Dry peptides were obtained (as TFA salt) by lyophilization and then verified by NMR and ESI-MS. On resin peptide F′GGGGG were synthesized using H-Rink Amide ChemMatrix Resin using the same method above. The F′GGGGG-functionalized resin was washed with DMF for 3 times and then left in D₂O/H₂O (for 3 days, change with fresh D₂O/H₂O each day). Then, the washed F′GGGGG resin was preserved in D₂O/H₂O in the fridge at 277 K.

### Nuclear Magnetic Resonance Spectroscopy (NMR)

1H, ¹³C & ¹⁹F NMR spectra of pure peptides were acquired in D₂O at 298 K on a Bruker AVANCE 400 (400 MHz) apparatus and a BrukerAVANCE 500 apparatus with TCI Cryoprobe (500 MHz). ¹H & ¹⁹F NMR spectra of other experiments (NMR titration, on-resin recognition tests) were acquired in phosphate buffer (D₂O) with pH 7.0 on a Bruker AVANCE 400 (400 MHz) apparatus. Chemical shifts were referenced to the residual solvent peak of HDO at 4.79 ppm.

### Isothermal Titration Calorimetry (ITC)

ITC experiments were operated on a Malvern MicroCal Auto-ITC200 apparatus at 298 K in 10 mM phosphate buffer with pH 7.0. In a of 1 injection of 0.6 μL and 32 consecutive injections of 1.2 μL with 90 s intervals between injections. The first data point was removed before analysis. The ITC data were fitted by MicroCal Analysis Centre software using one set of sites model. In a typical experiment for homoternary complexation, the host molecule (CB[8]) was loaded in the sample cell with a concentration of 0.1 mM, and the guest peptide was loaded in the syringe with a concentration of 2.0 mM. The injection and interval settings were the same as above. The first data point was removed from the data set before analysis. The obtained ITC curves were fitted by MicroCal Analysis Centre software using sequential binding model. All parameters were averaged with three replicates, s.d. n=3. Kₐ values <10² M⁻¹ were too low to be detected.

### Electrospray Ionization Mass Spectrometry (ESI-MS)

ESI-MS data were obtained on a Thermo Fisher Q Exactive Orbitrap mass spectrometry with a nanospraying ion source. Positive mode was chosen for all experiments at a working 35 temperature of 320° C. and a capillary voltage of 1.5 kV. All the sample solutions were prepared in pure water.

### Ultraviolet (UV) Spectroscopy

UV spectra were obtained on a Cary 4000 Ultravioletvisible Spectrometer. A pair of quartz cuvettes with 2 mm gap was used. Double beam mode with a fixed 2 nm spectral bandwidth and baseline correction were selected. In a typical run, 300 μl of 1 mM solution was transferred to the quartz cuvette and was scanned compared to phosphate buffer in D₂O. UV spectra were obtained in the linear absorption range (0.2-0.8) for better quantification.

### Confocal Microscopy

On-resin imaging was performed on a Leica Stellaris 5 confocal microscope (Leica Microsystems) with a 40× oil immersion objective and numerical aperture of 1.3. Suspensions of resin beads were dropped on a coverslip and stacks of 50 images with a resolution of 1024×1024 pixels at _EXC 405 nm were acquired. The stack boundaries were selected at the point where there was no distinguishable difference of signal-to-noise for both extremes (edges) of the beads. The 3D dataset was processed by Fiji/ImageJ and the maximum intensity Z-projection was obtained to compare the mean fluorescent intensity across resin beads. 3 groups of resin beads were tested: A group, original unmodified ChemMatrix resin (resin) and F′G5 functionalized resin (resin-G5F′); B group, resin-G5F′ with WGG but no CB[8](resin-G5F′+WGG), resin-G5F′ with WGG and CB[8](resin-G5F′+WGG+CB[8]), resin-G5F′ with WGG, CB[8] and then DMADA solution (resin-G5F′+WGG+CB[8]+DMAMA); C group, resin-G5F′ with WG5-dansyl but no CB[8](resin-G5F′+WG5-dansyl), resin-G5F′ with WG5-dansyl and CB[8](resin-G5F′+WG5-dansyl+CB[8]), resin G5F′ with WG5-dansyl, CB[8] and then DMADA solution (resin-G5F′+WG5-dansyl+CB[8]+DMAMA). 3 groups of resin beads were prepared according to the procedures in the UV Spectroscopy above. All samples then were drained and added with the same amount of phosphate buffer with pH 7.0 to yield suspensions (10 mM) for imaging.

### Circular Dichroism (CD) Spectroscopy

CD measurements were performed on a Chirascan spectrometer (Applied Photophysics). Scans were carried out at 298 K from 200-500 nm at 1.0 nm intervals. The acquisition time was 0.5 second in a 0.1 cm path length quartz cuvette. CD spectra were collected using solutions from the above UV experiments of the 8-day storage and release experiments. The original insulin solution was 0.2 mM. All solutions were diluted 2× before the measurements. All scans were collected and averaged out over three scans with background noise correction.

### Procedures for On-Resin Recognition

A standard procedure of NMR quantification for on-resin recognition within a peptide mixture is shown as follows. A solution of mixed peptides containing WGG, KGG, LGG, EGG, CB[8]([XGG]=CB[8]=1 mM) was used as the original cycle 0 solution. For cycle 1, 1.0 ml of the mixed peptides (1 mM) was added to the F′GGGGG-functionalized resin (10 mM) and shaken for 10 min at room temperature. After draining, 1 ml of 2 mM DMADA solution was added, shaken for 45 min, and collected as the released solution. For cycle 2, fresh CB[8](2.88 mg, 1.8 μmol) was added to 0.9 ml of the released solution (2 mM DMADA, 1.8 μmol) from cycle 1 to bind with excess DMADA and the isolated WGG. Around 0.1 ml solution was lost after each cycle. This new solution was added with the resin, shaken for 45 min, drained and then 0.9 ml of 2 mM DMADA solution was added to generate the released solution from cycle 2. For cycle 3, fresh CB[8](2.56 mg, 1.6 μmol) was added to 0.8 ml of the released solution (2 mM DMADA, 1.6 μmol) from cycle 2. This new solution was added with the resin, shaken for 45 min, drained and then 0.8 ml of DMADA solution was added to generate the released solution from cycle 3. The original cycle 0 solution and the subsequently released solutions from each cycle were tested with NMR. After NMR analysis, the relative amount of WGG was determined by integrating a set of doublet peak at 3.36 ppm; the relative amount of KGG was determined by integrating a set of triplet peak at 3.02 ppm; the relative amount of EGG was determined by integrating a set of triplet peak at 2.37 ppm. There was a singlet peak of KGG, LGG, EGG that overlapped at 3.95 ppm. Thus, the relative amount of LGG was determined by the relative amount of KGG and EGG subtracted from the integration of peak at 3.95 ppm.

A standard procedure of UV quantification for peptide on-resin recognition is shown as follows. Using a model tripeptide WGG as an example, 10 μmol F′GGGGG equivalent of resin D₂O suspension was added to a 12 ml syringe with a polyester frit (r=8.0 mm, h=2.0 mm). After D₂O was drained, 1 ml of 1 mM WGG:CB[8]=1:1 solution (original solution, in D₂O, phosphate buffer, pH=7.0) was added to the resin. Then, the plunge and a stopper were assembled to the syringe to form a sealed chamber. The syringe was transferred onto a wrist-action shaker and shaken for 10 min. Then, the liquid (resin-treated solution) in syringe was drained thoroughly and the resin was washed with D₂O once. After that, 1 ml of 2 mM DMADA solution (in D₂O, phosphate buffer, pH=7.0) was added to the resin and shaken for an additional 45 min. The solution (released solution) was drained for further testing. For efficiency test over 3 cycles (FIG. 4c), WGG:CB[8]=1:1 solution and DMADA solution were alternatively added to resin, shaken and drained for three cycles and produced a series of resin-treated and released solutions.

A standard procedure of UV quantification for insulin on-resin recognition is shown as follows. Insulin (Human Recombinant) was suspended at 0.2 mM using 0.22 μm filtered H2O. 1 N HCl was immediately added to reach acid concentration of 5 mM. Solution was gently stirred until fully dissolved and was filtered through 0.22 μm filter and stored at 277 K. Method was slightly modified from a Cold Spring Harbor Protocol (see Recipe: Insulin). Insulin solution was warmed to room temperature before being used in on-resin recognition. In a typical experiment, 8 μmol F′GGGGG equivalent of resin H2O suspension was added to a 12 ml syringe with a polyester frit (r=8.0 mm, h=2.0 mm). After H2Owas drained, 8 ml of 0.1 mM CB[8] solution was added to the resin and the plunge and a stopper were assembled to the syringe to form a sealed chamber. The syringe was transferred onto a wrist-action shaker and shaken for 12 hours. Then, after CB[8] solution was drained and resin was washed once with H2O, 0.8 ml of 0.2 mM insulin solution was added to the resin and shaken gently for 3 hours. Then, the liquid (resin-treated solution) in syringe was drained thoroughly and the resin was washed with H2Oonce. After that, 0.8 ml of 2 mM DMADA solution (in 5 mM of HCl) was added to the resin and shaken gently for an additional 3 hours. The solution (released solution) was drained. The origin insulin solution, resin-treated solution and released solution were measured on UV spectroscopy immediately.

The 8-day storage and release experiment was conducted as follows. 9 syringes were prepared each with 8 μmol F′GGGGG equivalent of resin H2Osuspension. On day 0, as the standard procedure of UV quantification for insulin on-resin recognition described above, all 9 syringes were drained, bound with CB[8], washed, bound with insulin, drained (to get resin-treated solutions), washed. All syringes with the resin were kept at room temperature. Starting from day 0 to day 8, on each day, a syringe with resin was added with 0.8 ml of 2 mM DMADA solution (to get released solution, one per day). All yielded solutions were analyzed on UV spectroscopy immediately. Numbers to calculate released percentage in FIG. 4g were absorbance at 276 nm.

### Synthesis & Characterization of Model Peptides

### (S)-2-amino-N-(2-((2-amino-2-oxoethyl)amino)-2-oxoethyl)-3-(perfluorophenyl) propenamide (F′GG)

¹H NMR (500 MHz, D₂O, ppm): 6=4.32 (t, 1H), 3.99 (q, 2H), 3.93 (s, 2H), 3.37 (d, 2H); ¹³C NMR (125 MHz, D₂O): 6=174.01, 170.89, 168.81, 146.39, 144.44, 141.90, 139.89, 138.49, 136.49, 107.29, 52.04, 42.30, 41.89, 23.85; ¹⁹F NMR (375 MHz, D₂O, ppm): δ=−142.65, -154.41, -162.18; ESI MS for [M-H]+: calc. m/z=369.10, found m/z=369.17.

### (S)—N-(2-amino-2-oxoethyl)-2-(2-aminoacetamido)-3-(perfluorophenyl) propanamide (GF′G)

¹H NMR (500 MHz, D₂O, ppm): δ=4.77-4.74 (m, 1H), 3.95-3.86 (m, 2H), 3.85-3.79 (m, 2H), 3.32 (dd, 1H), 3.20 (dd, 1H); ¹³C NMR (125 MHz, D₂O, ppm): δ=173.56, 172.10, 166.91, 146.25, 144.32, 141.27, 139.28, 138.29, 136.33, 109.48, 52.55, 42.00, 40.22, 24.43; ¹⁹F NMR (375 MHz, D₂O, ppm): δ=−143.48, −155.99, −162.89; ESI MS for [M-H]+: calc. m/z=369.10, found m/z=369.17.

### (S)-2-(2-(2-aminoacetamido)acetamido)-3-(perfluorophenyl)propanamide (GGF′)

¹H NMR (500 MHz, D₂O, ppm): δ=4.73-4.70 (m, 1H), 3.96 (s, 2H), 3.88 (s, 2H), 3.31 (dd, 1H), 3.16 (dd, 1H); ¹³C NMR (125 MHz, D₂O, ppm): δ=174.32, 170.86, 167.64, 146.27, 144.34, 141.17, 139.17, 138.24, 136.27, 109.89, 52.04, 42.05, 40.28, 24.52; ¹⁹F NMR (375 MHz, D₂O, ppm): δ=−143.47, −156.31, −163.04; ESI MS for [M-H]+: calc. m/z=369.10, found m/z=369.17.

### (S)-2-amino-N-(2-((2-amino-2-oxoethyl)amino)-2-oxoethyl)-3-(1H-indol-3-yl) propanamide (WGG)

¹H NMR (500 MHz, D₂O, ppm): δ=7.64 (d, 1H), 7.54 (d, 1H), 7.35 (s, 1H), 7.29 (t, 1H), 7.21 (t, 1H), 4.36 (t, 1H), 3.92 (d, 1H), 3.91-3.75 (m, 3H), 3.49-3.38 (m, 2H); ¹³C NMR (125 MHz, D₂O, ppm): δ=173.90, 171.22, 170.24, 136.11, 126.42, 125.23, 122.21, 119.61, 117.98, 112.03, 106.24, 53.68, 42.36, 41.83, 26.70; ESI MS for [M-H]+: calc. m/z=318.16, found m/z=318.34.

### (S)—N-(2-amino-2-oxoethyl)-2-(2-aminoacetamido)-3-(1H-indol-3-yl)propanamide (GWG)

¹H NMR (500 MHz, D₂O, ppm): δ=7.67 (d, 1H), 7.52 (d, 1H), 7.30-7.24 (m, 2H), 7.19 (t, 1H), 4.77-4.69 (m, 1H), 3.84 (d, 1H), 3.81-3.73 (m, 2H), 3.70 (d, 1H), 3.36-3.23 (m, 2H); ¹³C NMR (125 MHz, D₂O, ppm): δ=173.90, 173.77, 166.95, 136.04, 126.64, 124.54, 122.00, 119.38, 118.25, 111.89, 108.60, 54.89, 42.00, 40.23, 26.96; ESI MS for [M-H]+: calc. m/z=318.16, found m/z=318.34.

### (S)-2-(2-(2-aminoacetamido)acetamido)-3-(1H-indol-2-yl)propanamide (GGW)

¹H NMR (500 MHz, D₂O, ppm): δ=7.70 (d, 1H), 7.52 (d, 1H), 7.30-7.24 (m, 2H), 7.19 (t, 1H), 4.68 (dd, 1H), 3.99-3.87 (m, 2H), 3.77 (s, 2H), 3.34 (dd, 1H), 3.23 (dd, 1H); ¹³C NMR (125 MHz, D₂O, ppm): δ=176.11, 170.80, 167.56, 136.05, 126.84, 124.49, 121.89, 119.30, 118.29, 111.84, 108.86, 54.13, 42.11, 40.23, 26.96; ESI MS for [M-H]+: calc. m/z=318.16, found m/z=318.34.

### (S)-2-amino-N-(2-((2-amino-2-oxoethyl)amino)-2-oxoethyl)-3-phenylpropanamide (FGG)

¹H NMR (500 MHz, D₂O, ppm): δ=7.47-7.36 (m, 3H), 7.35-7.29 (m, 2H), 4.32 (t 1H), 4.01 (d, 1H), 3.94-3.87 (m, 3H), 3.29-3.20 (m, 2H); ¹³C NMR (125 MHz, D₂O, ppm): _=173.97, 171.27, 169.82, 133.68, 129.35, 129.14, 128.00, 54.41, 42.26, 41.90, 36.69; ESI MS for [M-H]+: calc. m/z=279.15, found m/z=279.34.

### (S)—N-(2-amino-2-oxoethyl)-2-(2-aminoacetamido)-3-phenylpropanamide (GFG)

¹H NMR (500 MHz, D₂O, ppm): δ=7.44-7.37 (m, 2H), 7.37-7.31 (m, 1H), 7.34-7.28 (m, 2H), 4.68 (t, 1H), 3.86 (t, 2H), 3.81-3.74 (m, 2H), 3.16 (dd, 1H), 3.07 (dd, 1H); ¹³C NMR (125 MHz, D₂O, ppm): δ=173.76, 173.49, 166.96, 136.05, 129.14, 128.80, 127.28, 55.32, 41.99, 40.18, 36.93; ESI MS for [M-H]+: calc. m/z=279.15, found m/z=279.34.

### (S)-2-(2-(2-aminoacetamido)acetamido)-3-phenylpropanamide (GGF)

¹H NMR (500 MHz, D₂O, ppm): δ=7.43-7.36 (m, 2H), 7.36-7.29 (m, 2H), 7.30 (t, 1H), 4.62 (dd, 1H), 3.94 (q, 2H), 3.85 (s, 2H), 3.19 (dd, 1H), 3.01 (dd, 1H); ¹³C NMR (125 MHz, D₂O, ppm): δ=175.70, 170.82, 167.57, 136.41, 129.14, 128.69, 127.11, 54.68, 42.01, 40.29, 36.98; ESI MS for [M-H]+: calc. m/z=279.15, found m/z=279.34

### (S)-2-(2-(2-aminoacetamido)acetamido)-3-(4-hydroxyphenyl)propanamide (GGY)

¹H NMR (500 MHz, D₂O, ppm): δ=7.20-7.13 (m, 2H), 6.90-6.83 (m, 2H), 4.56 (dd, 1H), 3.95 (q, 2H), 3.86 (s, 2H), 3.10 (dd, 1H), 2.94 (dd, 1H); ¹³C NMR (125 MHz, D₂O, ppm): δ=175.77, 170.79, 167.57, 154.37, 130.49, 128.22, 115.38, 54.84, 42.01, 40.30, 36.18; ESI MS for [M-H]+: calc. m/z=295.14, found m/z=295.34.

### (S)-2-amino-N-(2-((2-amino-2-oxoethyl)amino)-2-oxoethyl)-3-(naphthalen-2-yl) propanamide (NpGG)

¹H NMR (500 MHz, D₂O, ppm): δ=7.97 (t, 2H), 7.95-7.90 (m, 1H), 7.83-7.79 (m, 1H), 7.64-7.55 (m, 2H), 7.46 (dd, 1H), 4.44-4.39 (m, 1H), 3.89 (s, 2H), 3.74-3.62 (m, 2H), 3.44 (dd, 1H), 3.37 (dd, 1H); ¹³C NMR (125 MHz, D₂O, ppm): δ=173.74, 171.04, 169.72, 133.04, 132.38, 131.40, 128.84, 128.33, 127.72, 127.62, 126.92, 126.84, 126.61, 54.27, 42.23, 41.68, 36.87; ESI MS for [M-H]+: calc. m/z=329.16, found m/z=329.34.

### (S)—N-(2-amino-2-oxoethyl)-2-(2-aminoacetamido)-3-(naphthalen-2-yl)propanamide (GNpG)

¹H NMR (500 MHz, D₂O, ppm): δ=7.93 (td, 3H), 7.80-7.76 (m, 1H), 7.57 (tt, 2H), 7.46 (dd, 1H), 4.77-4.74 (m, 1H), 3.84 (dd, 2H), 3.73 (dd, 2H), 3.32 (dd, 1H), 3.23 (dd, 1H); ¹³C NMR (125 MHz, D₂O, ppm): δ=173.67, 173.45, 166.94, 133.76, 133.04, 132.10, 128.31, 127.75, 127.62, 127.47, 127.24, 126.62, 126.21, 55.25, 41.96, 40.16, 37.13; ESI MS for [M-H]+: calc. m/z=329.16, found m/z=329.34.

### (S)-2-(2-(2-aminoacetamido)acetamido)-3-(naphthalen-2-yl)propanamide (GGNp)

¹H NMR (500 MHz, D₂O, ppm): δ=7.97-7.89 (m, 3H), 7.79-7.75 (m, 1H), 7.57 (tt, 2H), 7.46 (dd, 1H), 4.73 (dd, 1H), 3.90 (q, 2H), 3.76 (s, 2H), 3.36 (dd, 1H), 3.16 (dd, 1H); ¹³C NMR (125 MHz, D₂O, ppm): δ=175.66, 170.78, 167.47, 134.17, 133.03, 132.06, 128.19, 127.71, 127.61, 127.49, 127.31, 126.51, 126.11, 54.53, 42.02, 40.21, 37.16; ESI MS for [MH]+: calc. m/z=329.16, found m/z=329.34.

### (S)-2,6-diamino-N-(2-((2-amino-2-oxoethyl)amino)-2-oxoethyl)hexanamide (KGG)

¹H NMR (500 MHz, D₂O, ppm): δ=4.12-4.02 (m, 3H), 3.95 (s, 2H), 3.02 (t, 2H), 2.01-1.90 (m, 2H), 1.73 (p, 2H), 1.55-1.43 (m, 2H); ¹³C NMR (125 MHz, D₂O, ppm): δ=05, 25.74; ESI MS for [M-H]+: calc. m/z=261.12, found m/z=261.34.

### (S)-2-amino-N-(14-amino-2,5,8,11,14-pentaoxo-3,6,9,12-tetraazatetradecyl)-3-(perfluorophenyl) propenamide (F′GGGGG)

¹H NMR (700 MHz, D₂O, ppm): δ=4.30 (t, 1H), 4.02 (m, 8H), 3.95 (s, 2H), 3.37 (d, 2H); ¹⁹F NMR (375 MHz, D₂O, ppm): δ=−142.63, −154.54, −162.22; ESI MS for [M-H]+: calc. m/z=540.16, found m/z=540.67.

### 2-amino-N-(14-amino-2,5,8,11,14-pentaoxo-3,6,9,12-tetraazatetradecyl)acetamide (GGGGGG)

¹H NMR (700 MHz, D₂O, ppm): δ=4.09 (s, 2H), 4.04 (d, 4H), 4.02 (s, 2H), 3.95 (s, 2H), 3.93 (s, 2H); ESI MS for [M-H]+: calc. m/z=360.16, found m/z=360.50.

### (S)-2-amino-N-(17-((5-(dimethylamino)naphthalene)-1-sulfonamido)-2,5,8,11,14-pentaoxo-3,6,9,12,15-pentaazaheptadecyl)-3-(1H-indol-3-yl)propenamiden(WGGGGG-dansyl)

¹H NMR (700 MHz, D₂O, ppm): δ=8.68 (d, 1H), 8.47 (d, 1H), 8.36 (d, 1H), 7.97-7.85 (m, 3H), 7.62 (d, 1H), 7.53 (d, 1H), 7.35 (s, 1H), 7.26 (t, 1H), 7.17 (t, 1H), 4.41 (t, 1H), 4.07 (s, 2H), 4.03 (s, 2H), 4.02-3.92 (m, 4H), 3.69 (s, 2H), 3.48-3.40 (m, 2H), 3.37 (s, 6H), 3.21 (t, 2H), 3.14 (t, 2H); ESI MS for [M-H]+: calc. m/z=765.31, found m/z=765.34.

### On-Resin Recognition of Insulin

As shown in FIG. 5a, under the same experimental conditions as FIG. 4f without using CB[8], insulin showed minimal absorption to F′GGGGG-resin. In FIG. 5b,c, by using the same ChemMatrix resin but with different peptide GGGGGG other than F′GGGGG loaded, regardless of the usage of CB[8], insulin exhibited negligible absorption in both cases. In FIG. 5d, even insulin and CB[8] were used at the same time, there was almost no absorption to blank resin (H-Rink Amide ChemMatrix resin, as purchased, without any loaded peptides). This confirmed that the absorption of insulin-CB[8] to F′GGGGG-resins was achieved by host-enhanced polar-πinteractions.

Comparing to the 77% efficiency of treating F′GGGGG resin with WGG (1.0 mM) solution, a higher efficiency of 94% is observed when treating F′GGGGG resin with insulin (0.2 mM) solution. This phenomenon is actually one of the advantages of on-resin insulin stabilisation. Considering the Ka values of WGG and FGG are quite close, the difference of absorption efficiency between WGG and insulin is not simply related to the equilibrium calculations. Unlike WGG, insulin has two relatively long peptide chains consisting of 51 amino acids, thus its relatively large size could make it kinetically trapped into the crosslinked PEG(material of ChemMatrix resin) resin network. This can increase the absorption efficiency to about 94%, which is considerably higher than free small tripeptide WGG.

TABLE 2
UV absorbance data at 276 nm for on-resin storage and on-demand
release of insulin on day 0, 2, 5, 8 of an 8-day experiment

| | insulin | insulin | insulin | insulin |
|---|---|---|---|---|
| | day 0 (abs) | day 2 (abs) | day 5 (abs) | day 8 (abs) |
| original | 0.23508 | 0.23508 | 0.23508 | 0.23508 |
| resin-treated | 0.01213 | 0.00845 | 0.01349 | 0.01162 |
| released | 0.17598 | 0.17676 | 0.16827 | 0.17394 |
| absorption | 94.8% | 96.4% | 94.3% | 95.1% |
| efficiency (%) | | | | |
| recycling | 100% | 100.4% | 95.6% | 98.8% |
| efficiency (%) | | | | |

Note:
^{a} The efficiency results for FIG. 4g were calculated from Table S8 using the following equations:

$$absorption {efficiency}_{day x} = 1 - \frac{{abs}_{resin - treated , day x}}{{abs}_{original , day x}}$$

$$recycling {efficiency}_{day x} = \frac{{abs}_{released , day x}}{{abs}_{original , day x}} , \frac{{abs}_{original , day 0}}{{abs}_{released , day 0}}$$

### Secondary Structure Verification of Released Insulin

As shown in FIG. 7 and Table 3, after 2, 5, 8 days of storage on functionalized resin, when released back into the solution phase, all three samples exhibited highly similar CD curves compared to freshly prepared insulin solution. This indicated that the secondary structures of insulin were intact after 2, 5, 8 days of storage under room temperature.

TABLE 3
CD absorbance data at 208 nm for on-resin storage and on-demand
release of insulin on day 2, 5, 8 of an 8-day experiment

| | insulin, | | | |
|---|---|---|---|---|
| | original | insulin, day 2 | insulin, day 5 | insulin, day 8 |
| | (θm · deg) | (θm · deg) | (θm · deg) | (θm · deg) |
| repeat 1 | −58.1 | −58.1 | −53.0 | −52.4 |
| repeat 2 | −55.2 | −55.4 | −48.9 | −52.3 |
| repeat 3 | −55.8 | −55.7 | −49.1 | −52.2 |
| signal | −56.4 | −56.4 | −50.3 | −52.3 |
| std dev | 1.5 | 1.5 | 2.3 | 0.1 |
| active | — | 100.0% | 89.2% | 92.7% |
| ratio | | | | |

### Additional Experimental

### On-Resin Selective Isolation of Aromatic Peptides

The release DMADA-CB[8] complex might have some weak interactions (i.e. electrostatic interactions, hydrogen bonds) with the remaining tripeptides, which could lead to some changes in chemical shifts after release. The yield of the selectively separated WGG for a single cycle was 77% (data not shown), so the final yield of WGG after 3 cycles of selectively separation was 77% x77% x77%=45.6% (see FIG. 8 also).

TABLE 4
¹H NMR integration analysis for on-resin recognition within a peptide mixture^{a}

| | WGG | | KGG | | EGG | | KGG + LGG + EGG | LGG | |
|---|---|---|---|---|---|---|---|---|---|
| | area | percentage (%) | area | percentage (%) | area | percentage (%) | total area | area | percentage (%) |
| cycle 0 | 1000.0 | 24.5 | 919.6 | 22.6 | 1129.5 | 27.7 | 3078.1 | 1029.0 | 25.2 |
| cycle 1 | 1000.0 | 72.7 | 108.8 | 7.9 | 175.3 | 12.7 | 376.1 | 92.0 | 6.7 |
| cycle 2 | 1000.0 | 86.7 | 25.6 | 2.2 | 40.5 | 3.5 | 153.2 | 87.1 | 7.6 |
| cycle 3 | 1000.0 | 95.0 | 16.4 | 1.6 | 25.4 | 2.4 | 52.7 | 10.9 | 1.0 |

Note:
^{a}All the above results of percentages were shown in FIG. 4d.

### REFERENCES

(1) Call et al. Cell 2006, 127, 355-368.
(2) Luo et al. Chem. Rev. 2016, 116, 13571-13632.
(3) Tang et al. Chem. Soc. Rev. 2014, 43, 7013-7039.
(4) Zhang et al. Nat. Chem. 2016, 8, 120-128.
(5) Crowley, P. B., Ed. Supramolecular protein chemistry; Monographs in supramolecular chemistry; The Royal Society of Chemistry, 2021; pp 1-312.
(6) Heitmann et al. J. Am. Chem. Soc. 2006, 128, 12574-12581.
(7) Logsdon et al. J. Am. Chem. Soc. 2011, 133, 17087-17092.
(8) Biedermann et al. Angew. Chem. Int. Ed. 2014, 53, 5694-5699.
(9) Clarke et al. J. Am. Chem. Soc. 2021, 143, 6323-6327.
(10) Wang et al. CCS Chem. 2021, 3, 1413-1425.
(11) Sonzini et al. Angew. Chem. Int. Ed. 2016, 55, 14000-14004.
(12) de Vink et al. Chem. Sci. 2021, 12, 6726-6731.
(13) Hou et al. Angew. Chem. Int. Ed. 2013, 52, 5590-5593.
(14) Li et al. Chem. Commun. 2017, 53, 5870-5873.
(15) Xu et al. ACS Appl. Mater. Interfaces 2017, 9, 11368-11372.
(16) Parkins et al. Biomaterials 2021,120919.
(17) de Vink et al. Angew. Chem. Int. Ed. 2017, 56, 8998-9002.
(18) Huang et al. J. Am. Chem. Soc. 2020, 142, 7356-7361.
(19) Huang et al. Nat. Mater. 2022, 21, 103-109.
(20) Filler et al. Nature 1965, 205, 1105-1105.
(21) Sogah et al. J. Am. Chem. Soc. 1979, 101, 3035-3042.
(22) Kataki-Anastasakou et al. J. Am. Chem. Soc. 2020, 142, 20513-20518.
(23) Huang et al. Langmuir 2016, 32, 12352-12360.
(24) Biedermann et al. J. Am. Chem. Soc. 2012, 134, 15318-15323.
(25) Zheng et al. Angew. Chem. Int. Ed. 2010, 49, 8635-8639.
(26) Pace et al. Angew. Chem. Int. Ed. 2012, 51, 103-107.
(27) Urbach et al. J. Chem. 2011, 51, 664-678.
(28) Patterson et al. Lancet 2009, 373, 2027-2033.
(29) Sluzky et al. Proc. Natl. Acad. Sci. U.S.A. 1991, 88, 9377-9381.
(30) Chinai et al. J. Am. Chem. Soc. 2011, 133, 8810-8813.
(31) Webber et al. Proc. Natl. Acad. Sci. U.S.A. 2016, 113,14189-14194.
(32) Maikawa et al. Nat. Biomed. Eng. 2020, 4, 507-517.
Kim et al. J. Am. Chem. Soc. 2000, 122, 540-541.
Recipe: Insulin (0.5 mg/mL). Cold Spring Harbor Laboratory Press, 2014.
Li et al. Chem. Commun. 2016, 52, 8537.

1. A ternary complex of a cucurbituril host with a first aryl guest and second aryl guest, wherein the first aryl guest is a group within a biomolecule, and the first and second aryl guests are different.

2. The complex of claim 1, wherein the cucurbituril host is CB[8] or a variant or derivative thereof.

3. The complex of claim 2, wherein the cucurbituril host is CB[8].

4. The complex of any one of the preceding claims, wherein the first aryl guest is selected from phenyl, naphthyl, such as naphth-2-yl, hydroxyphenyl, such as 4-hydroxyphenyl, and indolyl, such as indol-3-yl

5. The complex of any one of the preceding claims, wherein the second aryl guest is a fluorophenyl, such as perfluorophenyl.

6. The complex of any one of the preceding claims, wherein the second aryl guest is covalently linked, directly or indirectly, to a solid phase.

7. The complex of any one of the preceding claims, wherein the biomolecule is a polypeptide.

8. The complex of any one of the preceding claims, wherein the first aryl guest is provided within the side chain of a phenylalanine, tyrosine, indole or naphthylalanine residue.

9. The complex of any one of the preceding claims, wherein the biomolecule is insulin, such as human insulin, such as recombinant human insulin.

10. The complex of claim 9, wherein the first guest is the phenyl group within the phenylalanine residue at position 1 of insulin.

11. A method of decomplexing a complex according to any one of claims 1 to 10, the method comprising the step of treating the complex with a competitor guest, and permitting the competitor guest to displace at least the first aryl guest from the complex.

12. The method of claim 11, wherein the competitor guest is or comprises adamantane amine (ADA) or memantine (DMADA).

13. A method of forming a complex according to any one of claims 1 to 10, the method comprising the step of mixing a cucurbituril host together with a biomolecule having a first aryl guest, and a second aryl guest, and permitting the host and guests to self-assemble.

14. The method of claim 13, comprising the step of at least partially purifying the complex, such as by filtration.
